(19) United States
(12) Patent Application Publication
(10) Pub. No.: US 2024/0177866 A1
(43) Pub. Date: May 30, 2024

(54) KIDNEY HEALTH MONITORING SYSTEM

(71) Applicant: Welch Allyn, Inc., Skaneateles Falls, NY (US)

(72) Inventors: Craig M. Meyerson, Syracuse, NY (US); Gene J. Wolfe, Pittsford, NY (US); Eugene G. Urrutia, Apex, NC (US)

(73) Assignee: Welch Allyn, Inc., Skaneateles Falls, NY (US)

(21) Appl. No.: 18/525,595

(22) Filed: Nov. 30, 2023

Related U.S. Application Data
(60) Provisional application No. 63/429,124, filed on Nov. 30, 2022.

(51) Int. Cl. G16H 50/30; G16H 20/17

(52) U.S. Cl. CPC G16H 50/30; G16H 20/17

(57) ABSTRACT

An example method includes identifying physiological parameters of a patient and based on the physiological parameters of the patient and using at least one trained machine learning model, determining a metric indicative of kidney health of the patient. The example method further includes determining that the metric is outside of a predetermined range. In response to determining that the metric is outside of the predetermined range, the method includes outputting a recommendation based on the metric or administering a treatment to the patient based on the metric.

### CROSS-REFERENCE TO RELATED APPLICATION

This application claims priority to U.S. Provisional App. No. 63/429,124, which was filed on Nov. 30, 2022 and is incorporated by reference herein in its entirety.

### TECHNICAL FIELD

This application relates to medical devices and computing systems monitoring the kidney health of patients based on various physiological parameters.

### BACKGROUND

The kidneys are organs that perform essential blood filtration functions. Kidneys are responsible for removing waste products from the blood. In addition, kidneys perform fluid balancing functions. The kidneys remove these materials by siphoning them into urine. Although most people are born with two kidneys, many healthy people have only a single functional kidney.

However, many people experience a loss in kidney function known as kidney disease. According to the National Kidney Foundation, 33% of adults in the United States are at risk for kidney disease. In individuals with kidney disease, waste and excess fluid can build up in the blood stream, leading to a variety of serious symptoms. Individuals with kidney disease may require invasive treatments, such as dialysis. In serious cases, individuals may require kidney transplants for continued survival.

To prevent individuals from developing kidney disease, or from their kidney disease developing into a level only treatable by transplant, early detection of kidney disease and kidney disease progression is essential. Numerous laboratory tests, lifestyle factors, and other parameters contribute to kidney disease progression. These biomarkers are typically examined in isolation. Due to the large variety of factors impacting kidney disease progression, it is often difficult for patients and clinicians to track and predict kidney health over time. Because it is difficult to predict kidney health over time, patients and clinicians are unable to recommend or participate in early interventions that would prevent kidney health deteriorating into kidney failure.

### SUMMARY

Various implementations of the present disclosure relate to assessing and predicting the kidney health of a patient using a variety of different input parameters. In some cases, multiple physiological parameters of the patient are obtained. Using a predictive model and the physiological parameters, a kidney health score of the patient is calculated. This kidney health score, for instance, corresponds to the current health status of the patient, a likelihood that the patient will develop a kidney-related disease (e.g., kidney stones, kidney failure, etc.) within a predetermined time period, or some other actionable indicator of kidney health. In some cases, the kidney health can be output in a report. In some examples, the kidney health score can be used to identify various interventions, treatments, or lifestyle changes that can prevent further deterioration of kidney health.

Various types of devices can calculate the kidney health score. In some cases, the kidney health score is calculated by a device including at least one sensor configured to detect at least one of the physiological parameters. For example, a patient monitor may calculate the kidney health score. In some examples, a device configured to administer a treatment to the patient calculates the kidney health score. For example, the kidney health score may be calculated by a dialysis treatment device. In some implementations, a device may calculate the kidney health score outside of a clinical environment. For instance, a wearable device may monitor at least one physiological parameter and calculate the kidney health score. Alerts may be output to a clinician or the patient, directly. Thus, various implementations of the present disclosure enable multiple parties to monitor kidney health over time.

### DESCRIPTION OF THE FIGURES

The following figures, which form a part of this disclosure, are illustrative of described technology and are not meant to limit the scope of the claims in any manner.

FIG. 1 illustrates an example environment for tracking and optimizing kidney health of a patient.

FIG. 2 illustrates example signaling or training and/or utilizing a prediction system for tracking kidney health of a patient.

FIG. 3 illustrates an example of the input data that is input into one or more predictive models to generate a kidney health score.

FIG. 4 illustrates an example of the report generated based on input data and a kidney health score of a subject.

FIG. 5 illustrates an example kidney health report visually output on a display.

FIG. 6 illustrates an example process for generating a report indicating the kidney health of a patient.

FIG. 7 illustrates an example process for adjusting a treatment of a patient based on a kidney health score.

FIG. 8 illustrates at least one example device configured to enable and/or perform the some or all of the functionality discussed herein.

### DETAILED DESCRIPTION

Various implementations of the present disclosure will be described in detail with reference to the drawings, wherein like reference numerals present like parts and assemblies throughout the several views. Additionally, any samples set forth in this specification are not intended to be limiting and merely set forth some of the many possible implementations.

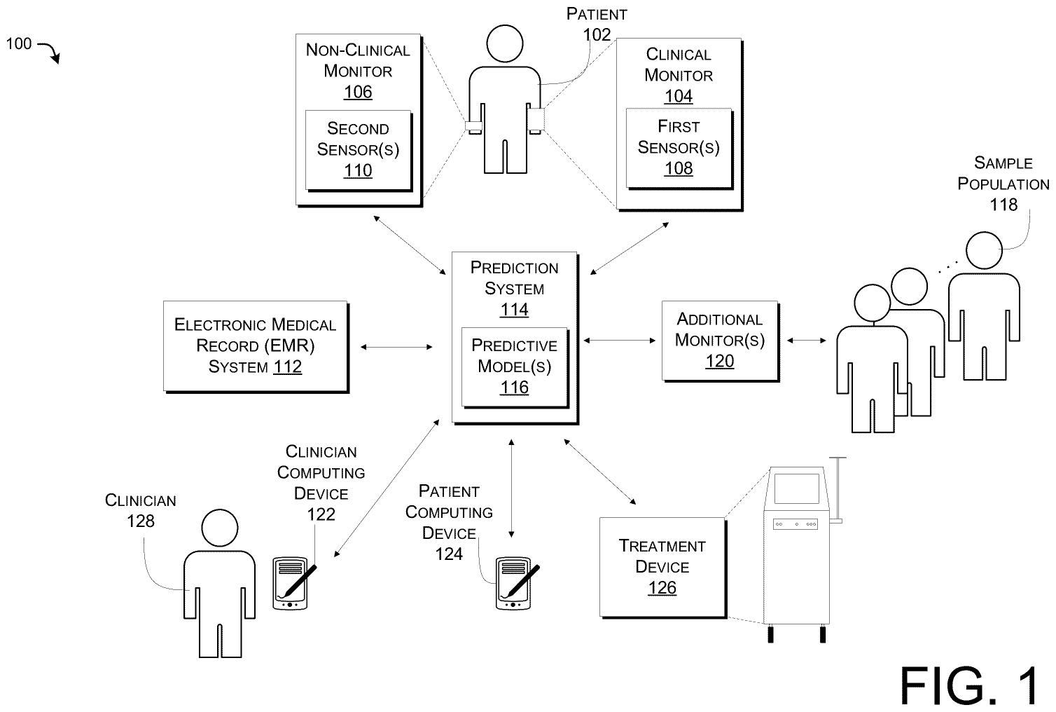

FIG. 1 illustrates an example environment 100 for tracking and optimizing kidney health of a patient 102. The patient 102, for instance, is a human or other type of animal subject. The patient 102, for example, has one or two kidneys. The kidney(s) are responsible for performing various filtering and fluid processing functions within the body of the patient 102. The kidney(s) receive blood as an input and emit blood and urine as outputs. The kidney(s) perform filtration, reabsorption, excretion, and secretion. In particular, the kidney(s) remove waste products (e.g., proteins, excess electrolytes, etc.) and water from the blood, and excrete the waste products and water in urine.

In various implementations a clinical monitor 104 and/or a non-clinical monitor 106 are configured to monitor the patient 102. In particular cases, the clinical monitor 104 includes one or more first sensors 108 configured to detect first physiological parameters of the patient 102 while the patient 102 is in a clinical environment (e.g., a hospital). In some implementations, the clinical monitor 104 is a hospital bed, vitals sign monitor, or a combination thereof. As used herein, the terms “bed,” “hospital bed,” and their equivalents, can refer to a padded surface configured to support a patient for an extended period of time (e.g., hours, days, weeks, or some other time period). For instance, the clinical monitor 104 includes a support structure that supports the weight of the patient 102 and the first sensor(s) 108 are integrated with the support structure. In some cases, the support structure includes railings that prevent the patient 102 from sliding off of a resting surface of the support structure. The railings may be adjustable, in some cases. According to various implementations, the clinical monitor 104 is configured to output alarms or otherwise communicate vital signs of patient 102 to external observers (e.g., care providers, visitors, and the like).

The non-clinical monitor 106, in various examples, includes one or more second sensors 110 configured to detect second physiological parameters of the patient 102 outside of a clinical environment (e.g., in a home of the patient 102). In addition, the environment may include an electronic medical record (EMR) system 112 that stores an EMR of the patient 102. The EMR, for instance, stores third physiological parameters of the patient 102.

As used herein, the terms “physiological parameter,” “parameter,” “patient parameter,” and their equivalents, can refer to a condition of an individual and/or the surrounding environment. In various implementations of the present disclosure, the first to third physiological parameters are relevant to the health of the kidney(s) of the patient 102. Examples of physiological parameters include blood pressure, respiratory rate, heart rate, pulse rate, urine output, glomerular filtration rate (GFR) (estimated and/or measured), sepsis risk score, body mass index (BMI), weight, medications consumed, age, body temperature (e.g., core temperature, oral temperature, or axillary temperature), the concentration of one or more substances in blood and/or serum, the concentration of one or more substances in urine, or any combination thereof. In various cases, the blood, serum, or urine of the patient 102 can be tested for a level of a protein (e.g., albumin, creatinine, or cystatin C), a gas (e.g., oxygen, nitrogen, or carbon dioxide, such as by detecting blood oxygenation), a sugar (e.g., glucose), an electrolyte (e.g., sodium, bicarbonate, potassium, chloride, phosphorus, calcium), or some other marker (e.g., inulin, iohexol, etc.). In some cases, a sample of blood, serum, or urine is removed from the patient 102 before it is tested. In some examples, the blood, serum, or urine is within the body of the patient 102 while it is tested. Various physiological parameters described herein can be detected in vitro and/or ex vivo.

GFR is often reported in volume (e.g., milliliters) of cleansed blood per time (e.g., minute) per body area (e.g., square meters), and can be estimated or directly measured. Estimated GFR (also referred to as “eGFR”) can be estimated based on a level of creatinine in the blood of the patient 102. For example, a blood sample is obtained from the patient 102 and the level of creatinine and/or cystatin C in the blood sample is determined. Because the kidneys are responsible for filtering out proteins, such as creatinine, from the blood, the level of creatinine is indicative of the patient's 102 kidney function. In some cases, the eGFR is also calculated based on an at least one of an age, biological sex, height, body composition (e.g., muscle mass), weight, or race of the patient 102.

Measured GFR (also referred to as “mGFR”) can be identified by administering an exogenous filtration marker to the patient 102 and measuring the level of the exogenous filtration marker in the patient's 102 blood, directly. The exogenous filtration marker, in various cases, is a substance that is exclusively filtered from the blood via glomerular filtration. Examples of exogenous filtration markers include, for instance, chromium 51-ethylenediamine tetraacetic acid, inulin, iohexol, iothalamate, and technetium 99m diethylenetriamine pentaacetic acid.

A sepsis risk score can be a metric indicative of the risk of the patient 102 developing sepsis in a predetermined time period. As used herein, the term “sepsis risk,” “sepsis risk score,” and the like, can refer to a likelihood that an individual will develop sepsis. For example, a sepsis risk can be represented by a percentage probability that that an individual will develop sepsis. In certain cases, a sepsis risk can also be associated with a confidence interval, may be limited to the likelihood that the individual will develop sepsis within a particular time period (e.g., one week), may be associated with a future treatment plan (e.g., the expected administration of antibiotics), or the like. In some cases, the sepsis risk score is determined based on the dwell time of an invasive device within the body of the patient 102, health conditions of the patient 102 (e.g., whether the patient 102 is immunocompromised), and other physiological parameters of the patient 102 (e.g., body temperature, blood pressure, heart rate, etc.).

The physiological parameters, in various cases, include one or more parameters reflective of current kidney function of the patient 102. For example, these parameters include the concentration of various elements in urea excreted by the patient 102, such as at least one of sodium, nitrogen, glucose, bicarbonate, potassium chloride, phosphorus, calcium, or albumin. Other parameters indicative of kidney function include GFR and urine output. These markers indicate whether there is current kidney disfunction of the patient 102.

In some cases, the physiological parameters include parameters reflective of a stress on the kidney(s) of the patient 102. For example, these physiological parameters include blood pressure, heart rate, body temperature, and sepsis risk score. Other physiological parameters that can result in kidney function degradation in the future include the concentration of various elements in the blood of the patient 102, such as sodium, nitrogen, glucose, bicarbonate, potassium chloride, phosphorus, calcium, albumin, creatinine, and oxygen (measured, e.g., as blood oxygenation). These parameters may be indicative of whether the kidney(s) of the patient 102 are likely to deteriorate in the future.

According to some examples, the physiological parameters include non-modifiable factors. For example, the age of the patient 102 may be a relevant physiological parameter that cannot be adjusted by clinical interventions or lifestyle changes by the patient 102. Other examples of non-modifiable factors include certain comorbidities of the patient 102. For example, the patient 102 may have a disease impacting kidney function, such as a genetic disease, renal agenesis, or diabetes. Examples of non-modifiable factors include historical information, such as previous diagnoses of the patient 102. For instance, if the patient 102 was previously diagnosed with amyloidosis, that diagnosis may be a non-modifiable factor.

Further, in some implementations, the physiological parameters include modifiable factors. These parameters may be adjustable based on clinical interventions and/or lifestyle changes by the patient 102. For instance, modifiable factors can be dependent on the diet of the patient 102 (e.g., whether the patient is on a low-sodium or low-protein diet), activity level of the patient 102, or stress management by the patient 102. Examples of modifiable factors include weight, BMI, heart rate, blood pressure, blood glucose, and the use of certain medications (e.g., NSAID use).

According to some implementations, the physiological parameters include one or more derived parameters. These derived parameters are calculated based on one or more other physiological parameters. Examples of derived parameters include, for instance, sepsis risk score, eGFR, and mGFR.

In some cases, the first sensor(s) 108 are configured to detect the first physiological parameters based on a sample obtained from the patient 102. For example, the first sensor(s) 108 may detect a level of cystatin C, inulin, iohexol, glucose, bicarbonate, potassium chloride, phosphorus, calcium, albumin, creatinine, or sodium in the blood and/or serum sample obtained from the patient 102. In various implementations, the first sensor(s) 108 detect the first physiological parameters based on a urine (urea) sample of the patient 102. For instance, the first sensor(s) 108 may detect a level of nitrogen, glucose, bicarbonate, potassium chloride, phosphorus, calcium, albumin, creatinine, or sodium in a urine sample obtained from the patient 102.

In some cases, the non-clinical monitor 106 is located outside of the clinical environment. For example, the non-clinical monitor 106 may include a home healthcare device and/or wearable device that can detect the second physiological parameters outside of the clinical environment. Examples of the non-clinical monitor 106 include wrist-wearable devices (e.g., a smart watch), portable blood glucose monitors, scales, pulse oximeters, and home vital sign monitors. In some cases, wrist-wearable devices are configured to detect a temperature, heart rate, or blood oxygenation of the patient 102. In some cases, a portable glucose monitor is configured to detect a blood glucose level of the patient. A scale may detect a weight (or body mass) of the patient 102. A home vital sign monitor, in some cases, may detect a temperature, blood oxygenation, heart rate, respiration rate, or any combination thereof.

The following table summarizes different types of devices that may be included in the first sensor(s) 108 and/or second sensor(s) 110:

| Physiological | |
|---|---|
| Parameter Detected | Sensor |
| Blood pressure | Blood pressure cuff |
| Respiratory rate | Chest distension sensor, processor(s) |
| | deriving respiratory rate from SpO2 |
| | waveform |
| Heart rate | Electrocardiogram (ECG) sensor |
| Pulse rate | processor(s) deriving pulse rate from SpO2 |
| | waveform |
| Blood oxygen concentration | SpO2 sensor, regional oximetry sensor |
| (e.g., blood oxygenation) | |
| Marker in blood, serum, | ELISA test strip and/or camera configured to |
| or urea | capture image of test strip; electrode(s) |
| | disposed in sample |
| Body temperature | Thermometer |
| Weight | Scale |

In various implementations of the present disclosure, a prediction system 114 evaluates the kidney health of the patient 102 based on the first to third physiological parameters. In various cases, the prediction system 114 stores one or more predictive models 116. The prediction system 114, for instance, may calculate a kidney health score based on the first to third physiological parameters and the predictive model(s) 116. In various implementations, the prediction system 114 generates the kidney health score in response to receiving a detected physiological parameter from the clinical monitor 104, the non-clinical monitor 106, the EMR system 112, or a combination thereof.

The kidney health score may be a metric indicative of the function and/or longevity of the kidney(s) of the patient 102. In particular examples, the kidney health score is based on a likelihood that the patient 102 will experience a kidney-related pathology within a predetermined time frame, such as within the next year, ten years, or the life of the patient 102. For instance, the kidney health score may be within a range of 0 (no likelihood that the patient 102 will experience the kidney pathology) to 100 (the patient 102 is currently experiencing the kidney pathology). Examples of kidney pathologies include kidney failure, kidney stones, and kidney cancer. The kidney health score may indicate the likelihood that the patient 102 will experience the kidney pathology and/or the severity of the kidney pathology experienced by the patient 102.

In some cases, the predictive model(s) 116 include one or more machine learning models. As used herein, the term “machine learning model,” and its equivalents, refers to a program, file, or collection of data that identifies patterns in a first dataset and/or predicts classifications of a second dataset based on the identified patterns. Examples of machine learning models include unsupervised learning models (principal component analysis models, kernel density estimation models, Gaussian mixture models, hierarchical clustering models, etc.), supervised learning models (e.g., gradient boosting models, decision trees, random forest models, naïve Bayesian models, support vector machines, etc.), semi-supervised learning models, self-supervised learning models (e.g., generative adversarial networks), In particular, the predictive model(s) 116 are defined by various model parameters that can be optimized to enable the predictive model(s) 116 to recognize patterns based on training data. The process of optimizing the model parameters can be referred to as “training.”

In some cases, the predictive model(s) 116 are trained by the prediction system 114. The prediction system 114 may receive and/or generate the training data. In some cases, the training data is based on measurements and/or health outcomes of a sample population 118 that omits the patient 102. For example, one or more additional monitors 120 may detect physiological parameters relevant to the kidney health of the sample population 118. Further, the EMR system 112 may include EMRs of the sample population 118. For example, the EMR system 112 may indicate whether members of the sample population 118 have experienced kidney failure, have undergone kidney transplants, have undergone dialysis treatments, or have other comorbidities (e.g., experienced sepsis) that are relevant to kidney function. The training data may include the physiological parameters of the sample population 118 and/or kidney health outcomes of the sample population 118. The additional monitor(s) 120 and/or EMR system 112 may indicate the training data to the prediction system 114.

In some implementations, the training data may include previous measurements of the physiological parameters of the patient 102 and/or previous kidney health scores of the patient 102. That is, the predictive model(s) 116 may be trained to specifically predict the kidney health score of the patient 102 based on individualized trends that are specific to the patient 102.

By training the predictive model(s) 116 based on both the sample population 118 and the patient 102 as an individual, the predictive model(s) 116 may more robustly predict the kidney health of the patient 102. For instance, the physiological parameters and EMRs of the sample population 118 may indicate that high blood pressure is associated with higher rates of kidney failure. The patient 102 may have transiently high blood pressure during regular jogs on Sundays. Without training the predictive model(s) 116 based on the physiological parameters of the patient 102, the predictive model(s) 116 could indicate that the patient 102 has a relatively poor kidney health score based on blood pressure readings on Sundays. However, by training the predictive model(s) 116 based on data indicative of the patient 102, the transient increase in blood pressure detected on Sundays could actually be recognized as indicative of an activity that will improve the kidney health score of the patient 102.

In particular examples, the kidney health score includes the probability of developing kidney failure within a predetermined time period, such as five years. For example, the kidney health score is the kidney failure risk score reported in Tangri et al., JAMA vol. 305, no. 15, pp. 1553-59 (2011), which is hereby incorporated by reference in its entirety. Tangri et al. describes using a Cox proportional hazards model with independence between predictors to predict kidney failure. Kidney failure was defined as the initiation of dialysis or kidney transplantation. Tangri et al. utilized the following physiological parameters to calculate the kidney failure risk score, including eGFR, biological sex, ACR, age, albumin, phosphorous, bicarbonate, and calcium.

According to some implementations, however, the kidney health score calculated by the predictive model(s) 116 is distinct from the kidney failure risk score reported by Tangri et al. For instance, unlike the kidney failure risk score of Tangri et al., in some cases, the kidney health score is calculated using one or more machine learning models. For instance, the predictive model(s) 116 include one or more machine learning models. In some cases, this enables the predictive model(s) 116 to identify more subtle patterns, relationships between physiological parameters, and evaluate a greater number of physiological parameters than the techniques reported by Tangri et al.

In some cases, the machine learning model(s) among the predictive model(s) 116 are trained exclusively based on training data gathered from patients that share a same condition as the patient 102. For example, if the patient 102 is undergoing dialysis, the training data is obtained exclusively from other patients undergoing dialysis; if the patient 102 is presenting to an emergency department, the training data is obtained exclusively from other patients that have presented to one or more emergency departments; if the patient 102 is in a medical-surgical (med-surg) unit in a hospital (e.g., the patient 102 is being prepared for and/or recovering from a surgery), the training data is obtained exclusively from other patients that are in one or more med-surg units in one or more hospitals. Limiting the training data to physiological parameters of patients with the same condition as the patient 102, in some cases, can enhance the accuracy of the kidney health score generated by the predictive model(s) 116.

According to some examples, the physiological parameters considered by the prediction system 114 are distinct from the risk factors described by Tangri et al. For example, in some cases, the prediction system 114 calculates the kidney health score exclusively based on vital signs and/or physiological parameters obtained in a basic metabolic panel. For instance, in some examples, the prediction system 114 is configured to generate the kidney health score without considering albumin, phosphorous, and calcium, which are not commonly available in ordinary clinical settings.

In particular cases, the predictive model(s) 116 include a gradient boosting machine. In particular examples, the physiological parameters input into the predictive model(s) 116 include vital signs (i.e., body temperature, heart rate, respiration rate, oxygen saturation, systolic blood pressure, diastolic blood pressure) and blood components identified in a standardized Renal Function Panel (i.e., glucose (mass/volume) in blood, urea nitrogen (mass/volume) in blood, creatinine (mass/volume) in blood, urea nitrogen/creatinine (mass ratio) in blood, calcium (mass/volume) in blood, sodium (moles/volume) in blood, potassium (moles/volume) in blood, chloride (mass/volume) in blood, carbon dioxide (moles/volume) in blood, and anion gap (moles/volume) in blood). Experimentally, it was determined that using a gradient boosting machine input with vital signs and blood components of the standardized Renal Function Panel showed high accuracy in predicting kidney failure and/or chronic kidney disease. For instance, it was determined that patients with kidney failure have lower blood pressure than other patients. Further, it was determined that patients with kidney failure exhibited higher creatinine, blood urea nitrogen, glucose, and anion gap than patients without kidney failure. In some implementations of the present disclosure, the gradient boosting machine can calculate a kidney health score using the vital signs and blood components of the standardized Renal Function Panel.

In various implementations, the prediction system 114 may output the kidney health score of the patient 102. In some cases, the prediction system 114 outputs a trend of the kidney health score over time. For instance, the prediction system 114 may output the kidney health score and/or trend to a clinician computing device 122, a patient computing device 124, a treatment device 126, the EMR system 112, or a combination thereof. According to some examples, the clinician computing device 122, the patient computing device 124, the treatment device 126, the EMR system 112, or a combination thereof, may store the kidney health score. For instance, the EMR system 112 may store the kidney health score in an EMR of the patient 102. In some implementations, the prediction system 114 may cause the clinician computing device 122, the patient computing device 124, the treatment device 126, or any combination thereof, to output the kidney health score and/or trend.

According to some implementations, the prediction system 114 generates a report based on the kidney health score. In various cases, the report includes a recommended management for the kidney health of the patient 102. For example, if the kidney health score is above a threshold, the report includes a recommended management to prevent further damage to the kidney(s) of the patient 102. Example management includes increasing a frequency of kidney health-related laboratory tests, dialysis, kidney transplant, medications, and other interventions.

In various implementations, the management is identified based on one or more modifiable factors of the patient 102. In some implementations, a modifiable factor of the patient 102 is a significant modifiable factor. For instance, the modifiable factor has a greater than threshold contribution on the kidney health score of the patient 102. For example, a relatively small modification in the physiological parameter (e.g., a five point reduction in systolic blood pressure) reduces the kidney health score by a significant amount (e.g., by ten points). The prediction system 114 may identify the significant modifiable factor by analyzing the kidney health score and/or the physiological parameters of the patient 102. The recommended management in the report, in some cases, includes one or more activities that can achieve the relatively small modification in the physiological parameter.

In various implementations, the report includes contextual information about the patient 102, such as in view of the sample population 118. For instance, if the patient 102 has a kidney health score consistent with 70 year-old members of the sample population 118, the report may indicate that the patient 102 has the kidney health of a 70 year old. In some cases, the contextual information may indicate whether a diagnostic event (e.g., a physical exam) and/or a treatment (e.g., kidney transplant) is a higher priority for the patient 102 than one or more additional patients. For example, the kidney health score of the patient 102 may exceed the kidney health scores of the additional patient(s).

The clinician computing device 122 may be operated by a clinician 128. As used herein, the term “computing device,” and its equivalents, may refer to a device including at least one processor configured to perform various operations. Examples of computing devices include servers, personal computers, Internet-of-Things (IoT) devices, certain medical devices, wearable devices, and mobile devices (e.g., tablet computers, smartphones, etc.). In some implementations, the clinician computing device 122 may receive and/or output the kidney health score, the trend, the report, or a combination thereof, from the prediction system 114. In some cases, the clinician computing device 122 receives a report that is specific to the functions of the clinician 128. For instance, if the clinician 128 is an intensive care unit (ICU) nurse with multiple patients, the report may indicate that the patient 102 has a lower priority for dialysis than one or more additional patients that the clinician 128 is managing. In some cases, if the clinician 128 is a primary care physician, the report may indicate a medication that the clinician 128 can prescribe to the patient 102 to help manage the kidney health of the patient 102.

The patient computing device 124 may be operated by the patient 102. The patient computing device 124 may be the non-clinical monitor 106, in some examples. For instance, the patient computing device 124 may be a wearable device (e.g., a smart watch). In some implementations, the patient computing device 124 receives a report with information that can be actionable by the patient 102. For example, if urine output is identified as a significant modifiable factor, the report may instruct the patient 102 to increase the amount of water that the patient 102 drinks per day. If blood pressure is identified as a significant modifiable factor, the report may instruct the patient 102 to exercise regularly, avoid dietary salt, or meditate consistently. If medication consumption is identified as a significant modifiable factor the report may instruct the patient 102 to avoid consuming NSAIDs. Thus, various implementations of the present disclosure can help the patient 102 take control of their own kidney health.

In various implementations, the treatment device 126 is configured to administer a treatment to the patient 102. The treatment, for instance, includes administration of a medication to the patient 102. For instance, the treatment device 126 may administer insulin to the patient 102, which may lower the blood glucose level of the patient 102. In some cases, the treatment device 126 may administer a medication (e.g., a beta blocker) to lower the blood pressure of the patient 102. In some examples, the treatment device 126 is configured to deliver the medication intravenously.

In some cases, the treatment includes hemodialysis and/or peritoneal dialysis. As used herein, the term “hemodialysis,” and its equivalents, may refer to a treatment by which a machine filters or otherwise purifies blood that has been removed from the body. For example, the treatment device 126 may be coupled to the circulatory system of the patient 102. A pump may bring blood from the patient 102 into a dialyzer. The dialyzer includes a dialysate solution (e.g., including water, sodium, potassium, calcium, magnesium, dextrose, acid, etc.) that is separated from the blood by a semi-permeable membrane. During hemodialysis, waste products (e.g., creatinine, urea, and water) from the blood are drawn across the semi-permeable membrane and into the dialysate solution. The processed blood is then transported back into the circulatory system of the patient 102. Thus, the treatment device 126, in some implementations, may reduce the concentration of waste products in the blood of the patient 102 by directly removing them. The treatment device 126, for instance, is configured to remove blood from the patient 102 (e.g., using one or more pumps), to generate the dialysate solution, to remove waste products from the blood (e.g., using a dialyzer), and to input the blood back into the patient 102 (e.g., using one or more pumps).

The term “peritoneal dialysis,” and its equivalents, may refer to a process that utilizes the peritoneum of a patient as a dialysis membrane. For example, a dialysate solution including solutes such as salts (e.g., sodium chloride), bicarbonate, lactate, and osmotic agents (e.g., glucose) is transported into the abdomen of the patient 102. The solution is disposed within the abdomen for a time period (referred to as a “dwell time”), during which waste products from the blood of the patient 102 diffuse from the blood vessels across the peritoneum. Subsequently, the fluid, including the waste products, is removed from the abdomen. In various implementations, the treatment device 126 is configured to generate the dialysate solution, input the dialysate solution into the abdomen of the patient 102 (e.g., using one or more pumps), remove the dialysate solution from the abdomen (e.g., using one or more pumps), or a combination thereof.

According to various implementations of the present disclosure, the treatment device 126 administers the treatment to the patient 102 based on the kidney health score. For example, the treatment device 126 may initiate the treatment in response to the kidney health score being above a predetermined threshold. In various cases, the treatment device 126 adjusts one or more treatment parameters that characterize the treatment based on the kidney health score. Examples of treatment parameters include medication dosage, concentration of one or more solutes in dialysate, dialysis flow rate, treatment time, dwell time, dialysis frequency, and others. Accordingly, the kidney health score generated by the prediction system 114 may be utilized to optimize the treatment.

Various components described with reference to FIG. 1 may be implemented by one or more computing devices, in hardware and/or software. For example, the prediction system 114 may be implemented in one or more on-prem or remote servers that are communicatively coupled to other elements of the environment 100. Various elements may be configured to communicate via one or more communication networks. As used herein, the term “communication network,” and its equivalents, may refer to one or more interfaces over which data can be transmitted and/or received. A communication network may include one or more wired interfaces (e.g., Ethernet, optical, or other wired interfaces), one or more wireless interfaces (e.g., BLUETOOTH; near field communication (NFC); Institute of Electrical and Electronics Engineers (IEEE)-based technologies, such as WI-FI; 3rd Generation Partnership Project (3GPP)-based technologies, such as Long Term Evolution (LTE) and/or New Radio (NR); or any other wireless interfaces known in the art). In various implementations, data is transmitted over an example interface via one or more Internet Protocol (IP) data packets and/or User Datagram Protocol (UDP) datagrams.

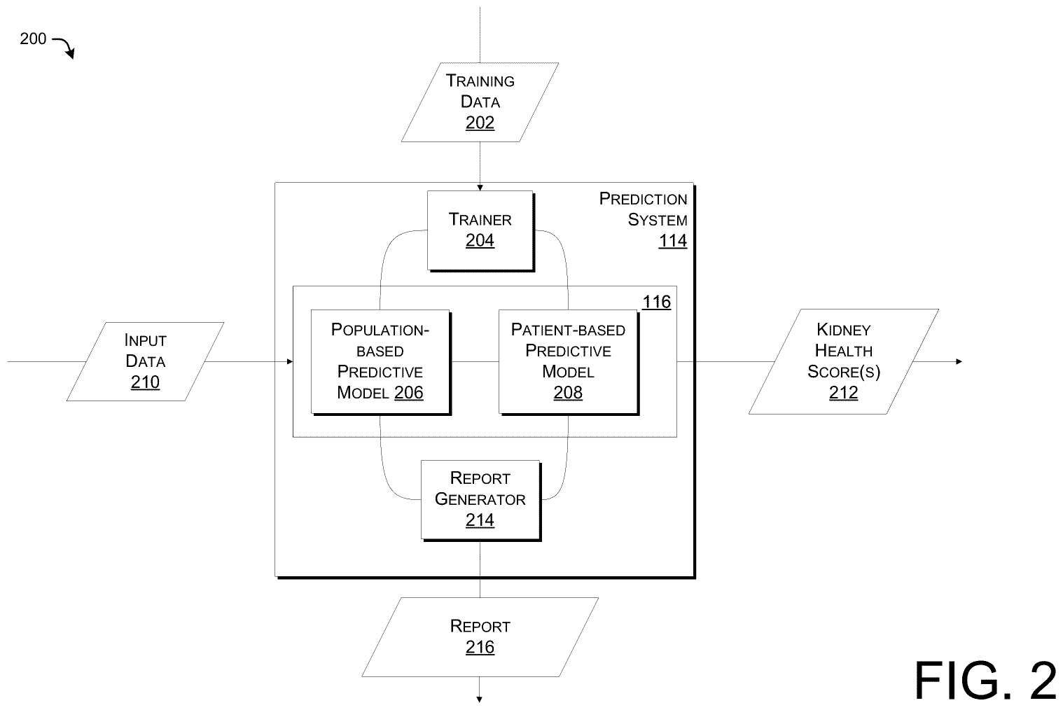

FIG. 2 illustrates example signaling 200 for training and/or utilizing the prediction system 114 described above with reference to FIG. 1. The prediction system 114, for example, can be executed and/or implemented by one or more computing devices, medical devices, or processors.

As shown, the prediction system 114 receives training data 202. In various implementations, the training data 202 includes physiological parameters of a subject (e.g., the patient 102) in addition to physiological parameters of a population omitting the subject (e.g., the sample population 118). Further, the training data 202 includes kidney health outcomes of the subject and the population. For example, the training data 202 may indicate whether each of the subject and members of the population are exhibiting kidney failure, have been diagnosed with a kidney-related health condition, have had a kidney transplant, have experienced kidney stones, have had a dialysis treatment, or the like. In some cases, the kidney health outcomes indicate current kidney health scores of the subject and members of the population. For instance, if a member of the population is in kidney failure, then the kidney health score of that member may indicate that the member has a 100% likelihood of having kidney failure.

A trainer 204 optimizes parameters of a population-based predictive model 206 and a patient-based predictive model 208 based on the training data 202. Specifically, the trainer 204 may train the population-based predictive model 206 based on the portion of the training data 202 corresponding to the population, and may train the patient-based predictive model 208 based on the portion of the training data 202 corresponding to the subject. The population-based predictive model 206 and the patient-based predictive model 208 may be connected to each other in series (e.g., outputs from one model are inputs to the other model), in parallel (e.g., the models share inputs), or a combination thereof.

In various implementations, the population-based predictive model 206 and the patient-based predictive model 208 are defined by parameters (e.g., numerical models). In some cases, the trainer 204 adjusts those parameters so that the population-based predictive model 206 and the patient-based predictive model 208 accurately output the kidney health outcomes in the training data 202 when they receive the parameters in the training data 202 as inputs. For instance, the trainer 204 may perform a training technique utilizing stochastic gradient descent with backpropagation, or any other machine learning training technique known to those of skill in the art, to optimize the parameters of the population-based predictive model 206 and the patient-based predictive model 208.

Once trained, the population-based predictive model 206 and the patient-based predictive model 208 can be used to assess the kidney health of the patient. For example, the predictive model(s) 116 receive input data 210 including one or more physiological parameters of the patient. In some cases, the input data 210 includes physiological parameters of the subject that were detected within a threshold time period (e.g., the last day, week, or month). For instance, the input data 210 may include a blood glucose reading obtained within the last minute, as well as a serum creatinine level obtained last week. In some cases, the input data 210 includes multiple measurements of the same physiological parameter, obtained at different times. For instance, the input data 210 may include heart rate measurements of the subject obtained at a sampling frequency of once an hour for the past day. The input data 210 is processed by the prediction system 114 using the population-based predictive model 206 and the patient-based predictive model 208. The predictive model(s) 116 may output a kidney health score 212 based on the input data 210. In various implementations, the kidney health score 212 indicates a likelihood that the subject will experience a kidney-related pathology (e.g., kidney stones, kidney failure, etc.) within a threshold period of time (e.g., the next day, week, month, or year). In some implementations, a treatment device adjusts a treatment administered to the subject (e.g., a medication dosage, a dialysis parameter, etc.) based on the kidney health score 212.

In some implementations, the prediction system 114 further includes a report generator 214. The report generator 214 may generate a report 216 based on the input data 210 and/or kidney health score 212. The report 216 may be output to a computing device operated by the subject and/or a clinician.

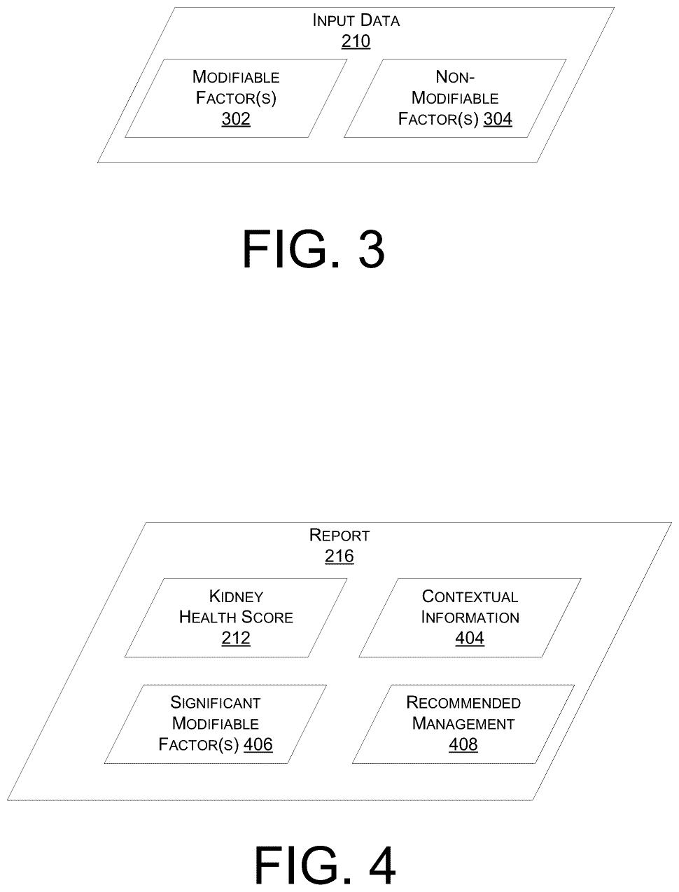

FIG. 3 illustrates an example of the input data 210 input into one or more predictive models to generate a kidney health score. The input data 210 includes physiological parameters of a subject. These physiological parameters include at least one modifiable factor 302 and at least one non-modifiable factor 304.

The modifiable factor(s) 302 include physiological parameters that can be changed by medical interventions and/or lifestyle changes. For example, the modifiable factor(s) 302 include weight, blood pressure, heart rate, pulse rate, medications consumed, activity level, diet, BMI, or any combination thereof.

The non-modifiable factor(s) 304 include physiological parameters that are difficult or impossible to change. For instance, the non-modifiable factor(s) 304 include height, age, eGFR, mGFR, sepsis risk score, body temperature, or any combination thereof.

Some physiological factors can, depending on the context, be part of the modifiable factor(s) 302 or the non-modifiable factor(s) 304. For instance, depending on the patient, respiratory rate can improve based on activity level or may be non-modifiable based on comorbidities (e.g., cystic fibrosis). In some cases, urine output can improve by drinking more water or may be non-modifiable in end-stage kidney disease. The level of a marker in blood, serum, or urea may be dependent on the diet of the patient, but may be uncontrollable in certain conditions.

FIG. 4 illustrates an example of the report 216 generated based on input data and a kidney health score of a subject. In various implementations, the report 216 can be output on a computing device operated by the subject or a clinician overseeing care of the subject. The report 216, for instance, includes the kidney health score 212, contextual information 404, at least one significant modifiable factor 406, and a recommended management 408.

In some cases, the report 216 includes the kidney health score 212 and/or a trend of the kidney health score 212 over time (e.g., the past month, year, five years, etc.). For example, the report 216 may include a graph showing the progression of the kidney health score 212 over a time period, such as the past year. Thus, the report 216 can communicate a current health of the kidney(s) of the subject and/or how the health of the kidney(s) has changed over time.

In some implementations, the report generator 214 compares the kidney health score 212 to the portion of the training data 202 reflecting the kidney health of the population, and generates the contextual information 404 based on the comparison. For instance, if the kidney health score 212 is indicative of a particular age group within the population, that age group may be indicated in the contextual information 404.

In some implementations, the report 216 includes one or more significant modifiable factors 406. For example, an entity generating the report 216 (e.g., the report generator 214) identifies modifiable factors among the physiological parameters of the subject used to generate the kidney health score 212. In some cases, the significant modifiable factor(s) 406 include at least one of those modifiable factors that is outside of a predetermined range. In some implementations, the entity generating the report 216 determines that a slight change (e.g., less than a threshold change) in at least one of the modifiable factors results in a significant change (e.g., greater than a threshold change) in the kidney health score 212. In various implementations, significant modifiable factor(s) 406 may suggest lifestyle changes that can result in substantially enhanced kidney health of the subject.

In various implementations, the recommended management 408 indicates one or more instructions or recommendations to enhance the kidney health of the subject. For example, the recommended management 408 may indicate a recommended medication or a recommendation that the subject discontinue a medication (e.g., an NSAID). In some implementations, the recommended management 408 may indicate one or more lifestyle changes, such as regular exercise, diet changes (e.g., following a low-protein, low-sodium, or low-sugar diet), water consumption goals (e.g., recommending that the patient drink 8 glasses of water a day), or maintaining a meditation practice. In some implementations, the entity generating the report 216 may identify the recommended management 408 based on the significant modifiable factor(s) 406. In some cases, the entity includes and/or has access to a datastore that stores various management recommendations in view of relevant significant modifiable factor(s) 406. For instance, the datastore may include an entry indexed by blood pressure (as a significant modifiable factor 406) that lists following a low-sodium diet as a recommended management 408.

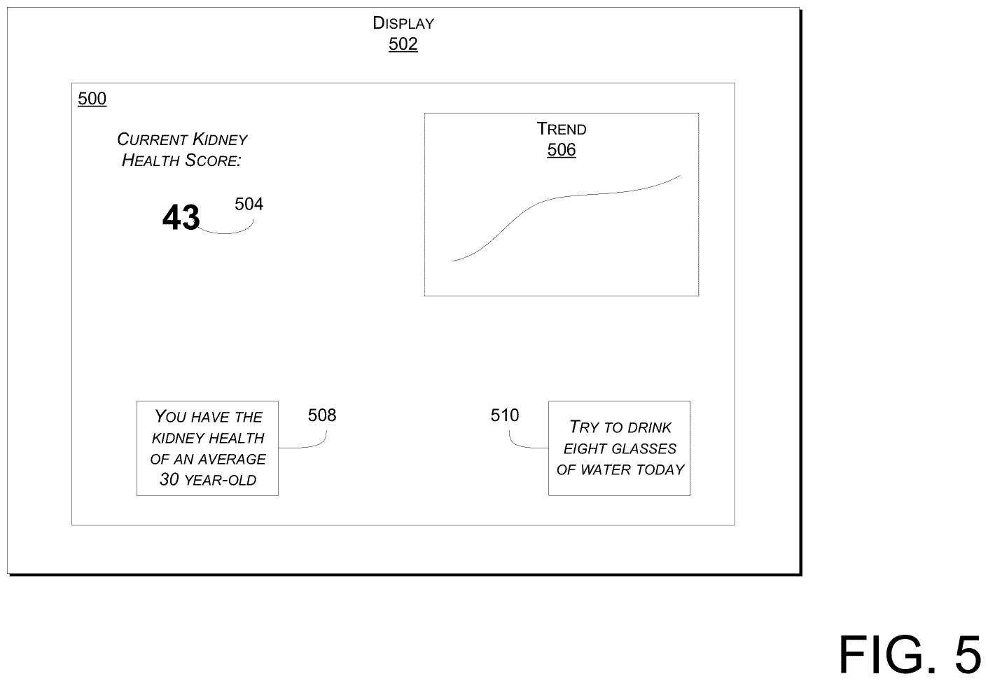

FIG. 5 illustrates an example kidney health report 500 visually output on a display 502. The display 502, for example, may be part of the patient computing device 124 described above with reference to FIG. 1. As shown, the report 500 includes a kidney health score 504, a trend 506 of the kidney health score over time, contextual information 508, and a recommended management 510.

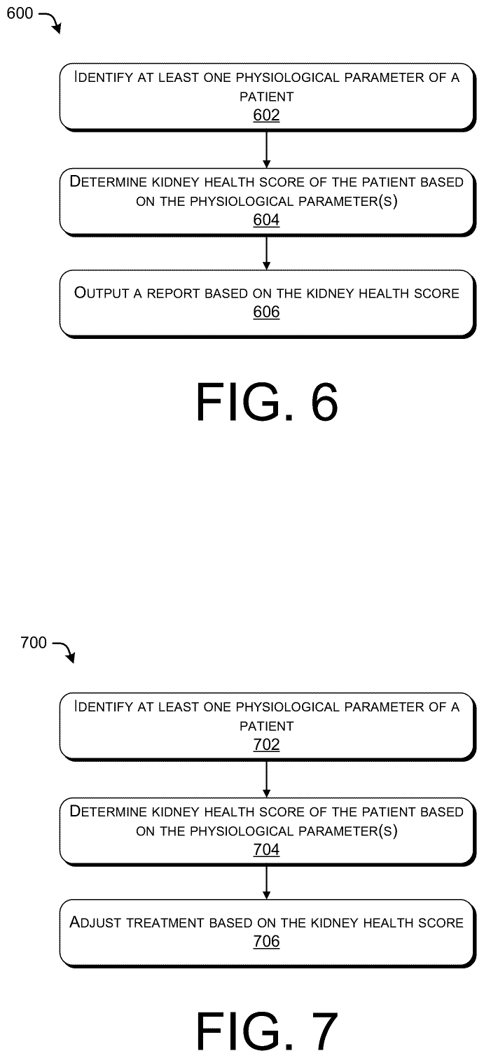

FIG. 6 illustrates an example process 600 for generating a report indicating the kidney health of a patient. The process 600 may be performed by an entity including a prediction system (e.g., the prediction system 114 described above), one or more processors, a computing device, a medical device, or any combination thereof.

At 602, the entity identifies at least one physiological parameter of a patient. The physiological parameter(s) may include at least one of diet, water consumption (e.g., within a predetermined time period), blood pressure, respiratory rate, heart rate, pulse rate, urine output, GFR (estimated and/or measured), sepsis risk score, BMI, weight, a medication consumed, an amount of a medication consumed (e.g., within a predetermined time period), age, body temperature (e.g., core temperature, oral temperature, or axillary temperature), the concentration of one or more substances in blood and/or serum, the concentration of one or more substances in urine, or any combination thereof. In some cases, the entity measures the physiological parameter(s) directly. In some examples, the entity receives an indication of the physiological parameter(s) from an external computing device. In various cases, an indication of the physiological parameter(s) is stored by the entity.

At 604, the entity determines a kidney health score of the patient based on the physiological parameter(s). In various implementations, the entity determines the kidney health score based on a predictive model including at least one trained machine learning model. For instance, the machine learning model may be trained based on previously detected physiological parameters of the patient and/or physiological parameters of a population including one or more subjects that omit the patient. In some cases, the machine learning model is also trained based on kidney health outcomes of the patient and/or population. In various implementations, the kidney health score is indicative of a likelihood that the patient will develop a kidney-related pathology within a predetermined time period. For example, the kidney health score may be based on a likelihood that the patient will develop kidney failure, kidney stones, or kidney cancer within the next 30 days, year, or ten years.

At 606, the entity outputs a report based on the kidney health score. In various examples, the report indicates the kidney health score. In some cases, the report includes one or more recommendations to improve the kidney health score of the patient. For example, the report indicates at least one significant modifiable factor. In some cases, the report indicates that the patient should change their weight, diet, activity level, water consumption, or should engage in stress management in order to improve their kidney health score. In some implementations, the report includes a recommended treatment, such as an indication that the patient should receive a dialysis treatment, a kidney transplant, or a medication.

FIG. 7 illustrates an example process 700 for adjusting a treatment of a patient based on a kidney health score. The process 600 may be performed by an entity including a prediction system (e.g., the prediction system 114 described above), one or more processors, a computing device, a medical device, a treatment device (e.g., the treatment device 126 described above), or any combination thereof.

At 702, the entity identifies at least one physiological parameter of a patient. The physiological parameter(s) may include at least one of diet, water consumption (e.g., within a predetermined time period), blood pressure, respiratory rate, heart rate, pulse rate, urine output, GFR (estimated and/or measured), sepsis risk score, BMI, weight, a medication consumed, an amount of a medication consumed (e.g., within a predetermined time period), age, body temperature (e.g., core temperature, oral temperature, or axillary temperature), the concentration of one or more substances in blood and/or serum, the concentration of one or more substances in urine, or any combination thereof. In some cases, the entity measures the physiological parameter(s) directly. In some examples, the entity receives an indication of the physiological parameter(s) from an external computing device. In various cases, an indication of the physiological parameter(s) is stored by the entity.

At 704, the entity determines a kidney health score of the patient based on the physiological parameter(s). In various implementations, the entity determines the kidney health score based on a predictive model including at least one trained machine learning model. For instance, the machine learning model may be trained based on previously detected physiological parameters of the patient and/or physiological parameters of a population including one or more subjects that omit the patient. In some cases, the machine learning model is also trained based on kidney health outcomes of the patient and/or population. In various implementations, the kidney health score is indicative of a likelihood that the patient will develop a kidney-related pathology within a predetermined time period. For example, the kidney health score may be based on a likelihood that the patient will develop kidney failure, kidney stones, or kidney cancer within the next 30 days, year, or ten years.

At 706, the entity adjusts a treatment based on the kidney health score. In some implementations, the entity causes a change in administration of a medication to the patient. In some implementations, the entity causes a dialysis treatment to be administered to the patient. In various cases, the entity changes a parameter of the treatment based on the kidney health score. For instance, the entity changes a concentration of a dialysate, a dosage of a medication, or a frequency of the treatment based on the kidney health score.

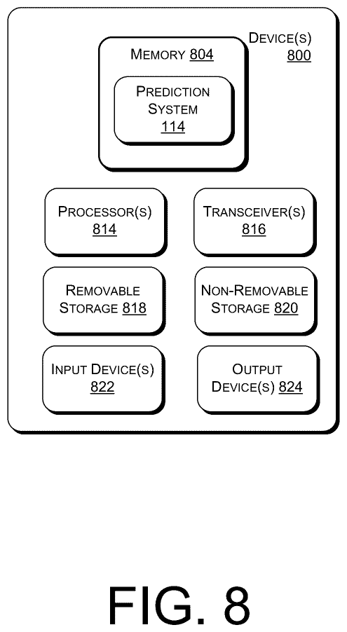

FIG. 8 illustrates at least one example device 800 configured to enable and/or perform the some or all of the functionality discussed herein. Further, the device(s) 800 can be implemented as one or more server computers 802, a network element on a dedicated hardware, as a software instance running on a dedicated hardware, or as a virtualized function instantiated on an appropriate platform, such as a cloud infrastructure, and the like. It is to be understood in the context of this disclosure that the device(s) 800 can be implemented as a single device or as a plurality of devices with components and data distributed among them.

As illustrated, the device(s) 800 include a memory 804. In various embodiments, the memory 804 is volatile (including a component such as Random Access Memory (RAM)), non-volatile (including a component such as Read Only Memory (ROM), flash memory, etc.) or some combination of the two.

The memory 804 may include various components, such as the prediction system 114. The prediction system 114 can include methods, threads, processes, applications, or any other sort of executable instructions. The prediction system 114 and various other elements stored in the memory 804 can also include files and databases.

The memory 804 may include various instructions (e.g., instructions in the prediction system 114), which can be executed by at least one processor 814 to perform operations. In some embodiments, the processor(s) 814 includes a Central Processing Unit (CPU), a Graphics Processing Unit (GPU), or both CPU and GPU, or other processing unit or component known in the art.

The device(s) 800 can also include additional data storage devices (removable and/or non-removable) such as, for example, magnetic disks, optical disks, or tape. Such additional storage is illustrated in FIG. 8 by removable storage 818 and non-removable storage 820. Tangible computer-readable media can include volatile and nonvolatile, removable and non-removable media implemented in any method or technology for storage of information, such as computer readable instructions, data structures, program modules, or other data. The memory 804, removable storage 818, and non-removable storage 820 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 800. Any such tangible computer-readable media can be part of the device(s) 800.

The device(s) 800 also can include input device(s) 822, such as a keypad, a cursor control, a touch-sensitive display, voice input device, etc., and output device(s) 824 such as a display, speakers, printers, etc. These devices are well known in the art and need not be discussed at length here. In particular implementations, a user can provide input to the device(s) 800 via a user interface associated with the input device(s) 822 and/or the output device(s) 824. The input device(s) 822 may include one or more sensors configured to detect one or more physiological parameters. In some cases, the output device(s) 824 are configured to administer a treatment, such as a medication and/or dialysis treatment.

As illustrated in FIG. 8, the device(s) 800 can also include one or more wired or wireless transceiver(s) 816. For example, the transceiver(s) 816 can include a Network Interface Card (NIC), a network adapter, a LAN adapter, or a physical, virtual, or logical address to connect to the various base stations or networks contemplated herein, for example, or the various user devices and servers. To increase throughput when exchanging wireless data, the transceiver(s) 816 can utilize Multiple-Input/Multiple-Output (MIMO) technology. The transceiver(s) 816 can include any sort of wireless transceivers capable of engaging in wireless, Radio Frequency (RF) communication. The transceiver(s) 816 can also include other wireless modems, such as a modem for engaging in Wi-Fi, WiMAX, Bluetooth, or infrared communication. In some implementations, the transceiver(s) 816 can be used to communicate between various functions, components, modules, or the like, that are included in the device(s) 800.

### EXAMPLE CLAUSES

1. A monitor, including: at least one processor; and memory storing: a predictive model including at least one trained machine learning model; and instructions that, when executed by the at least one processor, cause the at least one processor to perform operations including: generate, by applying physiological parameters of a subject to the predictive model, a kidney health score, the physiological parameters including a modifiable factor; determining that the kidney health score is outside of a predetermined range; and in response to determining that the kidney health score is outside of the predetermined range: determining that the modifiable factor is significant by determining that a predetermined change in the modifiable factor would cause the kidney health score to be inside of the predetermined range; identifying a management predicted to achieve the predetermined change in the modifiable factor; and outputting a report indicating the management.
2. The monitor of clause 1, wherein: the physiological parameters include at least one of an albumin level of the subject, a creatinine level of the subject, an albumin/creatinine ratio of the subject, a calcium level of the subject, a phosphorus level of the subject, a potassium chloride level of the subject, or a bicarbonate level of the subject; and the modifiable factor includes a blood glucose of the subject.
3. The monitor of clause 1 or 2, wherein the modifiable factor includes a blood glucose of the subject, a medication consumed by the subject, a diet of the subject, a water consumption of the subject, a blood pressure of the subject, a heart rate of the subject, a weight of the subject, or a BMI of the subject.
4. A method, including: identifying physiological parameters of a patient; based on the physiological parameters of the patient and using at least one trained machine learning model, determining a metric indicative of kidney health of the patient; determining that the metric is outside of a predetermined range; and in response to determining that the metric is outside of the predetermined range: outputting a recommendation based on the metric; or administering a treatment to the patient based on the metric.
5. The method of clause 4, wherein the parameters include at least one of blood pressure, respiratory rate, heart rate, pulse rate, urine output, estimated glomerular filtration rate (GFR), measured GFR, sepsis risk score, body mass index (BMI), weight, a medication dosage, age, body temperature, or a concentration of one or more markers in a fluid of the patient.
6. The method of clause 4 or 5, wherein the parameters include: at least one first parameter indicative of kidney function; and at least one second parameter indicative of kidney stress.
7. The method of one of clauses 4 to 6, wherein the parameters include at least one of a water consumption, a diet, or medication consumption.
8. The method of one of clauses 4 to 7, wherein identifying the physiological parameters of the patient includes: detecting, by a wearable device, at least one of the physiological parameters.
9. The method of clause 8, wherein the detecting at least one of the physiological parameters includes detecting, by the wearable device, a blood glucose level of the patient, a heart rate of the patient, a body temperature of the patient, or a blood oxygenation of the patient.
10. The method of one of clauses 4 to 9, wherein identifying the physiological parameters of the patient includes: detecting, by at least one sensor, at least one of the physiological parameters in a blood sample or a urea sample obtained from the patient.
11. The method of one of clauses 4 to 10, wherein identifying the physiological parameters of the patient includes obtaining multiple samples of a particular physiological parameter among the physiological parameters in a sampling period, and wherein determining the metric is based on the multiple samples.
12. The method of one of clauses 4 to 11, wherein identifying the physiological parameters of the patient includes receiving, from a sensor, data indicating a measurement of at least one of the physiological parameters, and wherein determining the metric is in response to receiving the data.
13. The method of one of clauses 4 to 12, wherein the metric is determined periodically.
14. The method of one of clauses 4 to 13, wherein the recommendation includes at least one of a numerical indicator of the metric or a graphical display of a trend in the metric over time.
15. The method of one of clauses 4 to 14, wherein the recommendation includes an instruction to administer a treatment to the patient.
16. The method of one of clauses 4 to 15, wherein the recommendation includes an instruction for the patient to engage in a lifestyle change.
17. The method of one of clauses 4 to 16, further including: identifying a modifiable parameter among the physiological parameters with greater than a threshold contribution to the metric, wherein the recommendation includes an instruction to adjust the modifiable parameter.
18. The method of one of clauses 4 to 17, wherein the recommendation includes an instruction to obtain an updated measurement of at least one of the physiological parameters.
19. The method of one of clauses 4 to 18, wherein the recommendation includes an instruction to obtain updated measurements of the at least one physiological parameters at a predetermined frequency.
20. The method of one of clauses 4 to 19, the physiological parameters being first physiological parameters detected at a first time, the metric being a first metric, the method further including: training the machine learning model by: identifying training data, the training data including: second physiological parameters of the patient, the second physiological parameters being detected at a second time; a second metric indicative of the health of the kidney at the second time; third physiological parameters of a population of subjects omitting the patient; and kidney health outcomes of the population of subjects; and optimizing model parameters of the at least one machine learning model using the training data.
21. A system including: at least one processor, and memory storing instructions that, when executed by the at least one processor, cause the at least one processor to perform the method of one of clauses 4 to 20.
22. A dialysis device, including: a treatment component configured to administer a dialysis treatment to a patient; at least one processor; and memory storing: a predictive model including at least one trained machine learning model; and instructions that, when executed by the at least one processor, cause the at least one processor to perform operations including: identifying physiological parameters of a patient; based on the physiological parameters of the patient and using the predictive model, determining a metric indicative of health of a kidney of the patient; and adjusting a treatment parameter characterizing the dialysis treatment based on the metric.
23. The dialysis device of clause 22, wherein the physiological parameters include a non-modifiable factor of the patient.
24. The dialysis device of clause 23, wherein the non-modifiable factor includes a previous diagnosis of the patient.
25. The dialysis device of one of clauses 22 to 24, wherein the physiological parameters include at least one modifiable factor of the patient.
26. The dialysis device of clause 25, wherein the modifiable factor includes at least one of a diet, a weight, a body mass index (BMI), a heart rate, a blood pressure, a blood glucose, or a medication usage of the patient.
27. The dialysis device of one of clauses 22 to 26, wherein the dialysis treatment includes a hemodialysis treatment or a peritoneal dialysis treatment.
28. The dialysis device one of clauses 22 to 27, wherein the treatment parameter includes a concentration of at least one solute in a dialysate.
29. The dialysis device of one of clauses 22 to 28, wherein identifying the physiological parameters includes retrieving at least one of the physiological parameters in an electronic medical record (EMR) of the patient.
30. The dialysis device of one of clauses 22 to 29, further including: a transceiver configured to receive the physiological parameters from at least one external device.
31. The dialysis device of one of clauses 22 to 30, further including: a sensor configured to detect at least one of the physiological parameters.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

As used herein, the term “based on” can be used synonymously with “based, at least in part, on” and “based at least partly on.”

As used herein, the terms “comprises/comprising/comprised” and “includes/including/included,” and their equivalents, can be used interchangeably. An apparatus, system, or method that “comprises A, B, and C” includes A, B, and C, but also can include other components (e.g., D) as well. That is, the apparatus, system, or method is not limited to components A, B, and C.

Although the subject matter has been described in language specific to structural features and/or methodological acts, it is to be understood that the subject matter defined in the appended claims is not necessarily limited to the specific features or acts described.

What is claimed is:

1. A monitor, comprising:
at least one processor; and
memory storing:
a predictive model comprising at least one trained machine learning model; and
instructions that, when executed by the at least one processor, cause the at least one processor to perform operations comprising:
generate, by applying physiological parameters of a subject to the predictive model, a kidney health score, the physiological parameters comprising a modifiable factor;
determining that the kidney health score is outside of a predetermined range; and
in response to determining that the kidney health score is outside of the predetermined range:
determining that the modifiable factor is significant by determining that a predetermined change in the modifiable factor would cause the kidney health score to be inside of the predetermined range;
identifying a management predicted to achieve the predetermined change in the modifiable factor; and
outputting a report indicating the management.

2. The monitor of claim 1, wherein:
the physiological parameters comprise at least one of an albumin level of the subject, a creatinine level of the subject, an albumin/creatinine ratio of the subject, a calcium level of the subject, a phosphorus level of the subject, a potassium chloride level of the subject, or a bicarbonate level of the subject; and
the modifiable factor comprises a blood glucose of the subject.

3. The monitor of claim 1, further comprising:
at least one sensor configured to detect at least one of the physiological parameters,
wherein the modifiable factor comprises a blood glucose of the subject, a medication consumed by the subject, a diet of the subject, a water consumption of the subject, a blood pressure of the subject, a heart rate of the subject, a weight of the subject, or a body mass index (BMI) of the subject.

4. A method, comprising:
identifying physiological parameters of a patient;
based on the physiological parameters of the patient and using at least one trained machine learning model, determining a metric indicative of kidney health of the patient;
determining that the metric is outside of a predetermined range; and
in response to determining that the metric is outside of the predetermined range:
outputting a recommendation based on the metric; or
administering a treatment to the patient based on the metric.

5. The method of claim 4, wherein the parameters comprise at least one of blood pressure, respiratory rate, heart rate, pulse rate, urine output, estimated glomerular filtration rate (GFR), measured GFR, sepsis risk score, body mass index (BMI), weight, a medication dosage, age, body temperature, or a concentration of one or more markers in a fluid of the patient.

6. The method of claim 4, wherein the parameters comprise:
at least one first parameter indicative of kidney function; and
at least one second parameter indicative of kidney stress.

7. The method of claim 4, wherein the parameters comprise at least one of a water consumption, a diet, or medication consumption.

8. The method of claim 4, wherein identifying the physiological parameters of the patient comprises:
detecting, by a wearable device, at least one of the physiological parameters.

9. The method of claim 8, wherein the detecting at least one of the physiological parameters comprises detecting, by the wearable device, a blood glucose level of the patient, a heart rate of the patient, a body temperature of the patient, or a blood oxygenation of the patient.

10. The method of claim 4, wherein identifying the physiological parameters of the patient comprises:
detecting, by at least one sensor, at least one of the physiological parameters in a blood sample or a urea sample obtained from the patient.

11. The method of claim 4, wherein identifying the physiological parameters of the patient comprises obtaining multiple samples of a particular physiological parameter among the physiological parameters in a sampling period, and
wherein determining the metric is based on the multiple samples.

12. The method of claim 4, wherein identifying the physiological parameters of the patient comprises receiving, from a sensor, data indicating a measurement of at least one of the physiological parameters, and
wherein determining the metric is in response to receiving the data.

13. The method of claim 4, wherein the recommendation comprises at least one of a numerical indicator of the metric or a graphical display of a trend in the metric over time.

14. The method of claim 4, wherein the recommendation comprises:
an instruction to administer a treatment to the patient; or
an instruction for the patient to engage in a lifestyle change.

15. The method of claim 4, further comprising:
identifying a modifiable parameter among the physiological parameters with greater than a threshold contribution to the metric,
wherein the recommendation comprises an instruction to adjust the modifiable parameter.

16. The method of claim 4, wherein the recommendation comprises an instruction to obtain an updated measurement of at least one of the physiological parameters at a predetermined frequency.

17. The method of claim 4, the physiological parameters being first physiological parameters detected at a first time, the metric being a first metric, the method further comprising:
training the machine learning model by:
identifying training data, the training data comprising:
second physiological parameters of the patient, the second physiological parameters being detected at a second time;
a second metric indicative of the health of the kidney at the second time;
third physiological parameters of a population of subjects omitting the patient; and
kidney health outcomes of the population of subjects; and
optimizing model parameters of the at least one machine learning model using the training data.

18. A dialysis device, comprising:
a treatment component configured to administer a dialysis treatment to a patient;
at least one processor; and
memory storing:
a predictive model comprising at least one trained machine learning model; and
instructions that, when executed by the at least one processor, cause the at least one processor to perform operations comprising:
identifying physiological parameters of a patient;
based on the physiological parameters of the patient and using the predictive model, determining a metric indicative of health of a kidney of the patient; and
adjusting a treatment parameter characterizing the dialysis treatment based on the metric.

19. The dialysis device of claim 18, wherein the dialysis treatment comprises a hemodialysis treatment or a peritoneal dialysis treatment.

20. The dialysis device of claim 18, wherein the treatment parameter comprises a concentration of at least one solute in a dialysate.
